(19) United States
(12) Patent Application Publication
(10) Pub. No.: US 2008/0249024 A1
(43) Pub. Date: Oct. 9, 2008

(54) Peptides facilitating or inhibiting a nterograde transport

(76) Inventor: Elaine L. Bearer, Providence, RI (US)

Correspondence Address: HELLER EHRMAN LLP, 4350 La Jolla Village Drive, 7th Floor, San Diego, CA, 92122, US

(21) Appl. No.: 11/975,388

(22) Filed: Oct. 18, 2007

Related U.S. Application Data
(60) Provisional application No. 60/852,896, filed on Oct. 18, 2006.

(51) Int. Cl. A61K 38/10; C07K 7/08; A61P 25/00; C12N 5/00

(52) U.S. Cl. 514/13; 530/326; 435/375; 514/14; 514/16; 530/327

(57) ABSTRACT

The present invention concerns amyloid precursor protein (APP)-based peptide sequences and their uses to facilitate or inhibit anterograde (synapse-directed) transport. The peptide sequences are of the formula GYENPTYX1X2X3X4X5X6X7X8 (SEQ ID NO: 13),
wherein
X1 is K, S, or R;
X2 is F or Y;
X3 is For L;
X4 is E or D;
X5 is missing or is Q or E;
X6 is M, V, S, or R;
X7 is Q, K or P;
X8 is missing or is N, E, A or I.

The invention further concerns JIP1/2-based peptides, and in particular peptides comprising or consisting of the peptide having the formula FVE YTC PTE DIY LE (SEQ ID NO: 14).

### CROSS REFERENCE TO RELATED APPLICATIONS

This is a non-provisional application claiming priority under 35 U.S.C. § 119(e) from U.S. provisional patent application No. 60/852,896, filed Oct. 18, 2006, the entire disclosure of which is expressly incorporated by reference herein.

### FIELD OF THE INVENTION

The present invention concerns peptide sequences and their uses to facilitate or inhibit anterograde (cell-surface and/or synapse-directed) transport.

### BACKGROUND OF THE INVENTION

Membrane-bound vesicles are primarily transported in neurons by fast axonal transport apparently on cytoskeletal tracks. Intracellular pathogens, such as herpes simplex virus (HSV) appear to co-opt this cellular transport machinery (Enquist, L. W., et al., (2002) Vet Microbiol 86, 5-16; Holland, D. J., et al., (1999) J Virol 73, 8503-11; Miranda-Saksena, M., et al., (2000) J. Virol. 74, 1827-39; Dohner, K., et al., (2005) Trends Microbiol 13, 320-7; and Bearer, E. L. & Satpute-Krishnan, P. (2002) Current Drug Targets—Infectious Disorders 2, 247-264) and thus may serve as tools to uncover the mechanisms governing cargo-motor interactions (Bearer, E. L. & Satpute-Krishnan, P., supra; Satpute-Krishnan, P., DeGiorgis, J. A. & Bearer, E. L. (2003) Aging Cell 2, 305-18).

HSV, a neurotropic virus that causes the recurrent cold sore, travels back and forth within neuronal processes at different stages in its life cycle (Roizman, B. & Knipe, D. M. (2001) in Fields Virology, eds. Knipe, D. M. & Howley, P. M. (Lippincott Williams & Wilkins, Philadelphia), Vol. 2, pp. 2399-2459). After the initial infection of the mucus membrane, HSV travels within the sensory nerve process to the trigeminal ganglion where it enters latency. Upon re-activation, newly synthesized viral particles are packaged in the cell body and then travel out to the periphery apparently by fast axonal transport within neuronal processes (Enquist, L. W., et al., (2002), supra; Holland, D. J., et al., (1999) supra; Miranda-Saksena, M., et al., (2000), supra; Ohara, P. T., et al., (2000) J Virol 74, 4776-86; Lycke, E., et al., (1984) J Gen Virol 65 (Pt 1), 55-64; Kristensson, K. et al., (1986) J Gen Virol 67 (Pt 9), 2023-8.).

Recently, HSV transport has been reconstituted in the giant axon of the squid in both directions—retrograde, as detergent-stripped particles (Bearer, E. L., et al., (2000) Proc Natl Acad Sci USA 97, 8146-50.), and anterograde (Satpute-Krishnan, P., et al. (2003), supra)—by injecting GFP-labeled virus into the giant axon of the squid. Viral particles transported in the anterograde direction were associated with a high copy number (˜3,000 molecules per virion) of amyloid precursor protein (APP) (Satpute-Krishnan, P., et al. (2003), supra). Because APP has been implicated as a motor receptor for transport in other systems (Gunawardena, S. & Goldstein, L. S. (2001) Neuron 32, 389-401; Kamal, A., et al., (2000) Neuron 28, 449-59; Kamal, A., et al., (2001) Nature 414, 643-8.12-14), it has become of interest whether APP might play a role in anterograde transport of virus.

Jun-kinase-interacting protein is a peripheral membrane protein that serves as scaffolding for Jun kinase (Verhey, K. J., et al., (2001) J Cell Biol 152, 959-70; Horiuchi, D., et al., (2005) Curr Biol 15, 2137-41; Muresan, Z. & Muresan, V. (2005) J Cell Biol 171, 615-25; Muresan, Z. & Muresan, V. (2005) J Neurosci 25, 3741-51). In yeast two-hybrid system, kinesin fished out JIP (Verhey, K. J., et al., (2001) J Cell Biol 152, 959-70) and in Drosophila, mutations in APIP.

### SUMMARY OF THE INVENTION

In one aspect the present invention concerns a peptide of the formula

| | |
|---|---|
| GYENPTYX1X2X3X4X5X6X7X8, | (SEQ ID NO: 13) |

wherein

### X1 is K, S, or R;

### X2 is F or Y;

### X3 is F or L;

### X4 is E or D;

X5 is missing or is Q or E;

### X6 is M, V, S, or R;

### X7 is Q, K or P;

X8 is missing or is N, E, A or I.

In one embodiment, in SEQ ID NO: 13 X1 is K, X2 is F, X3 is F, X4 is E, X5 is Q, X6 is M, X7 is Q and X8 is N.

In another embodiment, in SEQ ID NO: 13 X1 is K, X2 is Y, X3 is F, X4 is E, X5 is missing, X6 is M, X7 is Q and X8 is N.

In another aspect, the invention concerns a peptide of the formula FVE YTC PTE DIY LE (SEQ ID NO: 14).

In various embodiments, the foreging peptides are conjugated to a cargo, where conjugation includes linkage by any means, including direct fusion. The cargo can be an exogenous or endogenous cargo including, without limitation, cells, such as non-brain cells, peptides, polypeptides, non-peptide small molecules, nanoparticles such a cyclodextrin cages and other polymer-based non-viral and viral therapeutic gene vectors (Bergen et al., Pharm. Res. epubl. Oct. 12, 2007), and the like. In particular embodiments, the cargo is a brain permeable molecule and/or an adhesion molecule or a radiological contrast agents.

In another aspect, the invention concerns a method for facilitating anterograde neuronal transport of a single protein as cargo by conjugating the protein to a peptide of about 12 to about 17 amino acids, comprising the amino acid sequence of GYENPTY (SEQ ID NO: 1), and delivering the conjugate obtained along a neuronal pathway to a synapse.

In yet another aspect, the invention concerns a method for facilitating anterograde neuronal transport of a single protein as cargo by conjugating the protein to a peptide of the formula FVE YTC PTE DIY LE (SEQ ID NO: 14), and delivering the conjugate contained along a neuronal pathway to a synapse.

Fusion of the peptides to transmembrane proteins of intracellular systems can re-direct these systems. Redirection of mitochondria to synapses, for example, could improve energy metabolism during synaptic transmission. Redirecting lysosomes to cell surface could improve bacterial killing by polymorphonuclear leukocytes.

Cargoes and means of conjugation are as before, and in a preferred embodiment, the peptides comprise or have the sequence of SEQ ID NO: 13, where the symbols have the meanings described above, or comprise or have the sequence of SEQ ID NO; 14.

The diseases and conditions that can be treated by the methods of the present invention include neurodegenerative diseases, such as, for example, Alzheimer's disease, Huntington's disease, amyotrophic lateral sclerosis (ALS), siliary dyskinesias, retinitis pigmentosa, and Creutzfeldt-Jacob disease.

In a further aspect, the invention concerns a method for the treatment of a disease or condition benefiting from the inhibition of neuronal transport in a mammalian subject, comprising administering to the mammalian subject an effective amount of an inhibitor of the cytoplasmic C-terminus of the amyloid precursor protein (APP-C) of said mammalian subject.

In one embodiment, the inhibitor comprises the amino acid sequence of GYENPTY (SEQ ID NO: 1)

In another embodiment, the inhibitor comprises a peptide of the formula GYENPTYX1X2X3X4X5X6X7X8 (SEQ ID NO: 13), the variable symbols are as defined above.

In another embodiment the inhibitor comprises a peptide of the formula FVE YTC PTE DIY LE (SEQ ID NO: 14).

In other embodiments, the inhibitor is a peptide or non-peptide small molecule binding to APP-C or JIP1/2.

In a particular embodiment, the disease or condition benefiting from the inhibition of neuronal transport is Down's syndrome.

In a further aspect the invention concerns pharmaceutical compositions comprising the peptides of SEQ ID NO: 13, or SEQ ID NO: 14 or their conjugates to cargo molecules, in admixture with pharmaceutically acceptable ingredient(s).

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

Table 1. Diagram of APP showing location of peptide sequences and C99 construct used in this study.

Table 2. Comparison of velocities.

Table 3. Conservation of APP-C and APP.

FIG. 1. (a) Fast anterograde transport of APP-C beads in the giant axon. Time sequence (83 frames at 10 s intervals) of APP-C (red) and APP-N (green) beads co-injected into the squid giant axon begun ˜6.5 min after injection. Overlap of red and green fluorescence appears yellow at the injection site. (Supporting Video S1).

(b-c) Sequence-specific transport of APP-C beads. Two sequences (100 frames, 8 s intervals each) from the same axon co-injected with (b) green beads conjugated to the jumbled peptide APP-C^{J}, and (c) red beads conjugated to APP-C. The sequence with the APP-C^{J} green beads was captured first. (Supporting Videos S2 and S3).

FIG. 2. Squid APP is expressed in the giant axon and homologous to human/rat APP.

a) Western blot of rat brain extract (RB) and squid axoplasm (SA) probed in parallel for APP. Molecular weights indicated on left: 150, 100, 55, 45, 25 kDa.

b) Amino acid sequence comparison of the cytoplasmic domain of squid APP with human and rat. Identities with squid are highlighted. Antigenic epitope used to generate the antibody indicated by a bar above the sequence, aa 681-695.

FIG. 3. Analysis of individual APP-C bead movements.

a) During 240 s of this example (60-frame at 4 s intervals), 9 beads move and 6 remain stationary. Arrowheads mark two representative beads that travel in the anterograde direction past a group of stationary beads. (Supporting Video S4).

b) Tracings of a few bead tracks from (a) demonstrate linearity of movement. Bead tracks are indicated in a different color so that overlapping trajectories of individual beads can be followed during the time sequence of the video, stationary beads are black.

c) Tracings of a larger number of APP-C beads in another axon. APP-C beads travel exclusively in the anterograde direction with no reversals (100-frames at 4 s intervals). Bead tracks are colored as in (b).

FIG. 4. Analysis and comparisons of movements of C99-beads with various other bead conjugates and effect of APP-C peptide pre-injection.

a) Fast anterograde transport of C99-(red) and APP-C beads (green) in a co-injected axon (77 frames at 6 sec intervals). A green APP-C bead (arrow, lower three panels) follows the track of a red C99-bead (arrowhead, top three panels). The star in the upper left corner indicates stationary beads.

(b) Same video sequence as in (a) with the 77 frames superimposed showing the positions of beads as they traverse the field. Arrow indicates the starting point for the two beads that follow each other.

(c) Plot of distance moved per time comparing representative C99 (red) and APP-C (green) beads. Bead movements were measured in a 100-frame, 6 sec time-lapse video of a co-injected axon.

(d) Histogram of instantaneous velocities for C99 and APP-C with and without soluble peptide.

In all cases, the distance moved per frame was measured in 4 sec time-lapse videos for C99 (194 moves, 4 different beads), APP-C (227 moves, 6 beads), C99+peptide (49 moves, 3 beads). No moves were observed for APP-C with co-injected soluble peptide.

(e) Comparison of percent of beads that move for different bead conjugates with and without peptide. Comparable amounts of beads were injected for each experiment. An imaging field was selected at random within 500 μm of the injection site. Areas with large masses of beads were discarded. All beads appearing in each 100-frame video were counted. Moving beads were any that translocated >3 consecutive frames. The percent of moving beads was calculated for 3 different 100 frame video sequences and then averaged. As indicated, C99: 39.5%+/−4.5; C99+peptide: 10.3%+/−4.4; APP-C: 36%+/−6.2; APP-C+peptide: 0%; APP-680: 0%. Fewer C99 beads and no APP-C beads were found outside the injection site after peptide pre-injection. APP-680 beads were rarely found beyond the injection site and none moved.

(f) Plot of distance moved per time comparing C99 beads with and without pre-injection of APPC peptide. Shown are plots of two representative C99 beads moving in two different axons, one not injected with peptide (C99), and the other (C99+peptide) pre-injected with 500 pL of APP-C peptide at 2.5 mg/ml (0.66 picomoles). Each video sequence was 100 frames at 4 sec intervals.

FIG. 5. Squid APP amino acid sequence (SEQ ID NO: 10).

FIG. 6. JIP peptide-coated beads are transported in the axon. JIP peptide was conjugated to 100 nm fluorescent nanospheres (beads). Washed conjugated beads were injected into a freshly dissected giant squid axon and transport imaged by confocal microscopy. A typical injection site is shown in (A). The red JIP beads appear at the injection site at the beginning of confocal imaging, within 3 min of injection (0 min). At approximately an hour later, a stream of beads can be seen heading towards the synapse on the right side of the injection site (lower panel, 55 min). Higher magnification of the red JIP beads is shown in selected frames from a time-lapse series (B). Glycine-quenched green beads were co-injected and although a few appear beyond the injection site, these do not change their position over the 400 sec of this video sequence.

FIG. 7. Comparison of velocity of JIP and APP peptide beads and carboxylated negative charge beads in the giant axon.

FIG. 8. APP-C peptide and JIP peptide bind different motors. Shown is a Western blot of peptide beads and supernatants showing that beads conjugated with APP preferentially bind to kinesin 1 heavy chain, effectively clearing it from the brain extract as demonstrated by the absence of a kinesin 1 band in the supernatant lane of the APP bead pull down (upper panel). In contrast, JIP peptide preferentially pulls down kinesin 3 (Kif1a) from brain extract, removing all of this kinesin motor from the extract, as demonstrated by the absence of a band for kinesin 3 in the supernatant lane after JIP-bead exposure (lower panel).

### DETAILED DESCRIPTION OF THE INVENTION

### Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “axonal transport” is used herein to refer to directed transport of organelles and molecules along a nerve cell axon. The “axonal transport” can be “anterograde” (outward from the cell body) or “retrograde” (back toward the cell body). Thus, anterograde transport of membrane-bound organelles delivers molecules synthesized in the neuronal cell body outwards to distant synapses.

The term “exogenous cargo” is used herein to refer to any molecule that is not synthesized in the neuronal cell body from where it is transported, through axonal transport, to a target synapse.

The term “conjugate” or “conjugated” refers to any and all forms of linkage, and includes, without limitation, direct genetic or chemical fusion, and coupling though a linker or a cross-linking agent.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

As used herein, the terms “peptide,” “polypeptide” and “protein” all refer to a primary sequence of amino acids that are joined by covalent “peptide linkages.” In general, a peptide consists of a few amino acids, typically from about 2 to about 50 amino acids, and is shorter than a protein. The term “polypeptide” may encompass either peptides or proteins.

As used herein, the terms “Alzheimer's disease” or “AD” refer to a neurodegenerative disorder and encompasses all forms of Alzheimer's disease, including familial Alzheimer's disease and sporadic Alzheimer's disease. The term “familial Alzheimer's disease” refers to Alzheimer's disease that is associated with genetic factors, and thus demonstrates inheritance, while “sporadic Alzheimer's disease” refers to Alzheimer's disease that has no prior family history. Alzheimer's disease is generally characterized by progressive cognitive deterioration coupled with declining activities of daily living and neuropsychiatric symptoms and/or behaviors changes. Symptoms of Alzheimer's disease in a human subject can include, but are not limited to, loss of short term memory, which usually manifests itself as minor forgetfulness that becomes steadily more pronounced with illness progression, with relative preservation of older memories. As the disorder progresses, cognitive (intellectual) impairment extends to the domains of language, skilled movements, recognition, and those functions closely related to the frontal and temporal lobes of the brain (such as decision making and planning). Hallmark pathologies within brain tissue include extracellular neuritic P-amyloid plaques, neurofibrillary tangles, neurofibrillary degeneration, granulovascular neuronal degeneration, synaptic loss, and extensive neuronal cell death.

The term “early-onset Alzheimer's disease” is used herein to Alzheimer's disease diagnosed to occur before the age of 65 in humans. As used herein, the term “late-onset Alzheimer's disease” refers to Alzheimer's disease is diagnosed to occur after the age of 65 in humans.

A disease or condition “benefiting from the facilitation of neuronal transport” is any disease or condition the pathology of which includes impairment in the neuronal, such as axonal, transport. Without limitation, such diseases and conditions include neurodegenerative disease, such as Alzheimer's disease, Huntington's disease, amyotrophic lateral sclerosis (ALS), siliary dyskinesias, retinitis pigmentosa, and Creutzfeldt-Jacob disease.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented.

### DETAILED DESCRIPTION

Fast anterograde transport of membrane-bound organelles delivers molecules synthesized in the neuronal cell body outwards to distant synapses. Identification of the molecular “zipcodes” on organelles that mediate attachment and activation of microtubule-based motors for this directed transport is a major area of inquiry. The present invention is based, at least in part, on the identification of a short peptide sequence (15aa) from the cytoplasmic C-terminus of amyloid precursor protein (APP-C) sufficient to mediate the anterograde transport of peptide-conjugated beads in the squid giant axon. APP-C beads travel at fast axonal transport rates (0.53 μm/s average, 0.9 μm/s maximal velocity) while beads coupled to other peptides co-injected into the same axon remain stationary at the injection site. This transport appears physiologic, as it mimics behavior of endogenous squid organelles and of beads conjugated to C99, a polypeptide containing the full-length cytoplasmic domain of APP. Beads conjugated to APP lacking the APP-C domain are not transported, suggesting that the soluble peptide competes with protein-conjugated beads for axoplasmic motor(s). Co-injection of APP-C peptide reduces C99 bead motility by 75% and abolishes APP-C bead motility. The APP-C domain is conserved (13/15aa) from squid to human and peptides from either squid or human APP behave similarly. Thus the present invention provides a conserved peptide “zipcode” sufficient to direct anterograde transport of exogenous cargo and suggest that one of APP's roles may be to recruit and activate axonal machinery for endogenous cargo transport.

The “zipcode” of the present invention comprises the core sequence GYENPTY (SEQ ID NO: 1), and is typically about 12 to about 17 amino acids long. In particular embodiments, the peptide of the invention are 13-17, 14-17, 15-17, 16-17, 15-16, 14-16, 13-16, or 13-15 amino acids long. In a preferred embodiment, the peptide of the present invention are 15 amino acids long.

In a particular embodiment, the “zipcode” peptides herein are encompassed by the formula GYENPTYX1X2X3X4X5X6X7X8 (SEQ ID NO: 13),

wherein

### X1 is K, S, or R;

### X2 is F or Y;

### X3 is F or L;

### X4 is E or D;

X5 is missing or is Q or E;

### X6 is M, V, S, or R;

### X7 is Q, K or P;

X8 is missing or is N, E, A or I.

The invention is further based on the identification of another peptide, derived from the Jun Kinast Interacting Protein, JIP 1/2 that recruits a different microtubule-based motor. This peptide has or comprises the formula FVE YTC PTE DIY LE (SEQ ID NO: 14), recruits kinesin 3 (Kif1a), and directs transport of peptide conjugated beads outwards from the cell center to the synapse.

The peptides herein can be conjugated to an endogenous or exogenous cargo, which can be delivered to a distant synapse by neuronal (axonal) transport.

Conjugation can be performed by any methods known in the art. Thus, techniques for coupling molecules to amino acids are well known to those of skill in the art. Such methodologies may be found in standard chemical text books and publications, such as, for example, U.S. Pat. No. 5,876,727; WO 99/61054; Isomura, S. et al. J. Org. Chem. 66:4115 4121 (2001); Matsushita, H. et al. Biochem. Biophys. Res. Comm. 57:1006 1010. (1974); Langone, J. L. and Van Vunakis, H., Methods Enzymol. 84:628 640 (1982); Wong, Chemistry of Protein Conjugation and Cross-Linking. CRC Press, Inc., Boca Raton, Fla. (1991.) Conjugates, such as fusion proteins generated by genetic engineering may also be used to “:tag” other proteins or glycoproteins with these peptides for directed delivery.

The peptides of the present invention can be used to facilitate anterograde neuronal transport of a cargo, such as an exogenous cargo that can be used for the treatment of diseases or conditions the pathology of which involves an element of a defective anterograde neuronal transport. Without limitation, such diseases and conditions include neurodegenerative diseases, such as Alzheimer's disease, Huntington's disease, amyotrophic lateral sclerosis (ALS), siliary dyskinesias, retinitis pigmentosa, and Creutzfeldt-Jacob disease.

An additional condition that can be treated according to the present invention is Down's syndrome, which may befenit from inhibiting excess transport, for example, by delivery of a peptide of the present invention, or by treating a patient with an inhibitor, such as a small molecule inhibitor, of the endogenous APP-C.

The peptides of the present invention can also be used to engineer cells to display surface antigens or for subsequent discovery research into processes requiring or required by anterograde transport. Thus, redesigning the cell surface might enable the use of extracellular molecules to kill or mutate or re-direct that cell's behavior. In one example, direction of proteins to the cell surface allows the placement of exogenous (ie, designed) susceptibility epitopes on the cell surface for subsequent killing by chemotherapeutic agents.

Finally, peptide zipcodes can be used to trace neuronal circuits and their activity in disease states or for discovery research.

Methods of delivery, the determination of dosages and similar parameters are well known in the art and are well within the skill of those of ordinary skill in the pertinent art. Thus, delivery of the peptides of the present invention may be performed by a variety of means, including, for example, direct injection, delivery with viral vectors, or by gene therapy.

Further details of the invention are illustrated by the following non-limiting examples.

### EXAMPLE 1

### Identification of a Peptide Zipcode Sufficient for Anterograde Transport within the Amyloid Precursor Protein

### A. Materials and Methods

Dissection and Microinjection of the Giant Axon of the Squid

The giant axon was obtained from squid (Loligo pealei) at the Marine Biological Laboratory, Woods Hole, Mass. (46). Red and green beads, each conjugated to a different peptide (1.5 nl at 1012-109 beads/ml), were loaded into a mercury micropipette either as a mixture or in equal volumes separated by oil (Bearer, E. L., et al., (2000) Proc Natl Acad Sci USA 97, 8146-50; Terasaki, M., S. et al., (1995) Proc Natl Acad Sci U S A 92, 11500-3; Jaffe, L. A. & Terasaki, M. (2004) Methods Cell Biol 74, 219-42, FIG. S1). For each pair of beads the order of injection was alternated such that each color would have an opportunity to recruit motors first.

### Peptide Conjugation to Fluorescent Microspheres

Carboxylated microspheres (beads, 0.1 μm diameter) with red (580/605 nm) and green (505/515 nm) fluorescence (Invitrogen) were washed through a Low Binding Durapore filter (100 nm cut-off, Millipore). Uncoated beads were diluted in motility buffer (Brady, S. et al., (1982) Science 218, 1129-31) and injected without further treatment.

Custom synthesized peptides (Aves Labs, Inc) were cross-linked to beads via their amino terminus to allow presentation of the carboxy end of the peptide to the cytoplasm, mimicking the physiological orientation of APP. APP constructs (C99 and APP-680) were generated by PCR from human APP-YFP in pShuttleCMV (E.-M. Mandelkow and Jacek Biernat, Max Planck Institute, Hamburg, Germany (Stamer, K., V. et al., (2002) J Cell Biol 156, 1051-63)). APP-YFP is actively transported in neurons (Stamer et al., supra). For C99, the YFP moiety was replaced with mRFP (Campbell, R. E., et al., (2002) Proc Natl Acad Sci USA 99, 7877-82.) and for APP-680 with GFP. Each construct was cross-linked to a bead of similar spectrum as the fluorescent protein, and conjugated beads individually checked for monochromic emission by confocal. C99 beads displayed only red and no green fluorescence, and APP-680 beads were only green with no red. Accuracy of all sequences was confirmed by sequencing. Recombinant protein was generated in PC12 cells and purified by anti-fluorescent protein affinity columns (Vector Labs). Protein concentrations were determined by Bio-Rad protein assay, and composition and purity determined by SDS-PAGE.

Peptides or recombinant proteins (20 μl of a 2 mg/ml stock) were mixed in 300 μl of 50 mM MES buffer with 10 μl of 2% beads for 15 min. Cross-linker (1-(3-dimethylaminopropyl)-3-ethylcarbodiimide hydrochloride; EDAC, Molecular Probes) was added to a concentration of 10 μg/ml and the solution adjusted to pH 6.5 with 1M NaOH. After 5 hr the reaction was quenched by adding 2M glycine for a final of 0.1M. Conjugation was confirmed by analyzing peptide concentrations before and after by protein assay. Protein:bead ratio was adjusted that equivalent molar amounts of each protein would be conjugated to the beads. Efficiency of conjugation was relatively similar for all peptides. Binding sites on the beads (10⁶) were saturated by using 300-1000 fold excess peptide. For C99 and APP-680, protein concentration was adjusted so that 3,000 copies per bead were conjugated to the surface and unoccupied sites blocked with glycine.

### Imaging Axonal Transport by Confocal Microscopy

Detection parameters were set to ensure that each color was uniquely detected in its appropriate channel by imaging beads on coverslips with the Zeiss LSM 510 Laser Scanning Confocal Microscope. Fluorescent bead movements in the axon were collected with 10× Plan Neofluor 0.3NA air, and 40× Achroplan 0.8NA water correctible objectives. Green and red fluorescence and phase images were collected simultaneously using Zeiss LSM510 multi-tracking.

### Analysis of Transport

Rates and trajectories of APP-C beads were measured by stepping through the frames in either the Zeiss LSM browser or NIH ImageJ (http://rsb.info.nih.gov/nih-image/). Only particles moving into and out of a frame were included. Rates of moving particles were statistically analyzed and graphed using Microsoft Excel.

### Cloning and Sequencing of Squid APP

Squid APP was cloned as part of a project to acquire a database of genes expressed in the squid nervous system. Extracted mRNA from squid stellate ganglia was reverse transcribed by oligo-dT priming. Double-stranded cDNA was directionally ligated into pCMVSport6 vector (Invitrogen). Resultant clones were sequenced using universal primers from the 5′ end. Blastp search of predicted amino acid sequences from these clones identified one homologous to human APP.

### Results

### The C-Terminal Domain of APP is Sufficient to Mediate Anterograde Transport

Initial experiments focused on whether a peptide containing the conserved GYENPTY (SEQ ID NO: 1) motif, implicated in a variety of protein-protein interactions (De Strooper, B. & Annaert, W. (2000) J Cell Sci 113 (Pt 11), 1857-70), might be sufficient to mediate transport of exogenous cargo within the squid giant axon. This peptide, termed APP-C, represents the final 15 amino acids of the 47 amino acid C-terminus of APP which extends into the cytoplasm where it has access to cytoplasmic motors. A second peptide, APP-N, corresponding to a domain in the extracellular N-terminus, was used as a negative control as this region of APP is internal to the organelle and thus should have no ability to facilitate transport.

APP-N and APP-C peptides were each conjugated to a different color of fluorescent bead, and the behaviors of the two types of beads compared when co-injected into the same axon. The stability of a co-injected oil droplet was monitored as a sign of axonal integrity. In all experiments two colors of beads, each conjugated to a different peptide, were co-injected. A peptide was determined to lack transport capability only when tested in axons where directed transport of other particles could be observed.

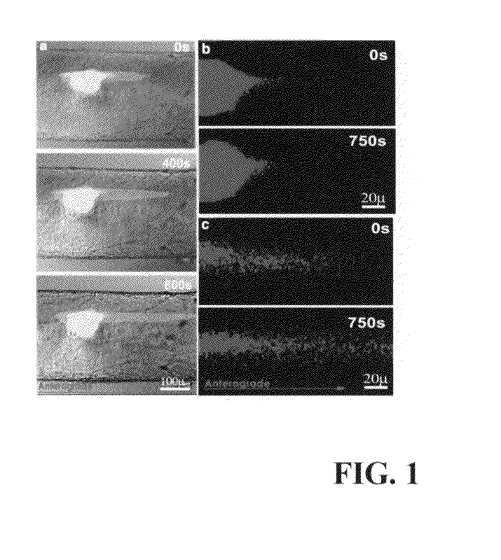

Beads conjugated to APP-C began transport immediately upon injection and already formed a plume heading towards the synapse by the time (1-3 min) the axon was transferred to the confocal microscope (FIG. 1a, red beads). The rate of advance of the plume was calculated by measuring the increase in intensity along a line of pixels drawn from the injection site to the edge of the video frame. The leading edge of plumes advanced over a 1 mm distance at fast axonal transport rates from 0.33 μm to 0.4 μm/s (n=5). Individual beads conjugated to APP-C displayed fast transport capability (0.41+/−07 μm/sec instantaneous, and 0.9 maximum velocities, n=227). These transport rates are similar to those of endogenous organelles and exogenous particles transported by active transport in squid and other invertebrate axons (Table 2).

As expected, APP-N beads remained stationary at the injection site for more than an hour of observation (FIG. 1a, green beads) while co-injected APP-C beads were rapidly transported past them (FIG. 1a, red beads). Unconjugated carboxylated beads undergo transport when injected into invertebrate axons (Adams, R. J., Bray, D. (1983) Nature 303, 718-720; Terasaki, M., Schmidek, A., Galbraith, J. A., Gallant, P. E. & Reese, T. S. (1995) Proc Natl Acad Sci USA 92, 11500-3). Thus conjugation of the APP-N peptide to beads masks the carboxylic residue and inhibits this transport.

To test whether electrostatic charge or sequence of APP-C was responsible for this transport, we synthesized a peptide with the same amino acid composition as APP-C but in jumbled order, APP-C^{j}. This peptide had the same charge but not the same sequence as APP-C. APP-C^{j} conjugated to beads with the same efficiency as APP-C. Co-injection of APP-C^{j} with APP-C beads revealed that APP-C^{j}-beads remained near the injection site while adjacent APP-C beads were rapidly transported (FIG. 1b, green APP-C^{j} and c, red APP-C). Rare individual APP-C^{j} beads found beyond the injection site were not seen to move during imaging sessions. Thus the transport of APP-C was sequence-specific and not a consequence of electrostatic charge.

Next, it has been tested whether other domains from the cytoplasmic tail of APP were also capable of mediating transport. Synthetic peptides designed to span the cytoplasmic domain of APP (Table 1) were coupled to beads and co-injected with APP-C beads into axons. Beads conjugated to equivalent amounts of peptides from other regions of the cytoplasmic domain were never seen to move, even though adjacent APP-C beads were rapidly transported. The difference between APP-C and any other peptide was robust and reproducible, occurring in more than 40 different axons independent of injection order or of bead color.

A significant number of APP-C beads were transported. When 10⁶ APP-C beads were injected, ˜0.5 to 1.3×10³ leave the injection site per minute. In comparison, only ˜0.001×10³ APP-N beads were observed to leave the injection site per minute. None of the beads conjugated to the jumbled peptide were observed to leave the injection site during 10 min of recording time in three axons in which adjacent APP-C beads were transported. Similar low rates were observed for beads conjugated to all peptides except APP-C. At this rate, it would take up to 2,000 min for all 10⁶ APP-C beads to leave the injection site. To determine if all APP-C beads could leave the injection site, we injected the smallest number of beads that could be imaged successfully, 0.5×10³. Under these conditions, individual beads left the injection site in single file, with approximately half of the beads transported out within 20 min of injection, for a rate of ˜12.5 beads/min.

Squid APP is Expressed in Axons and also Sufficient for Cargo Transport

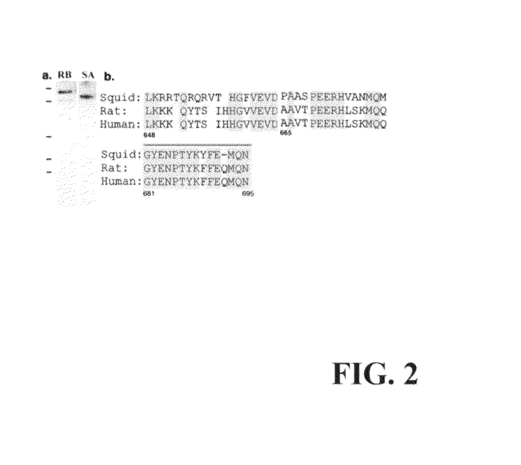

Squid APP was cloned from the stellate ganglion that contains the cell bodies that give rise to the giant axon. The predicted amino acid sequence is 612 aa long (SEQ ID NO: 10) with greater identities to human APP (38% identity) than to the other human homologues, APLP1 (32%) and APLP2 (34%) (Acc. No. DQ913735). The cytoplasmic domain (SEQ ID NO: 11) is 64% identical to human and rat which are 100% identical to each other (SEQ ID NO: 12; FIG. 2b). The squid APP-C domain was nearly identical to rat/human (13/15 aa identity). Anti-peptide antibodies against the conserved C-terminus that recognize rat brain APP also detected the squid protein which migrates at slightly lower molecular weight in SDS-PAGE (FIG. 2a).

Peptides spanning the 47aa cytoplasmic domain of squid APP were synthesized (Table 1). These peptides behaved similarly to those derived from the human sequence. Only the APP-C peptide was uniquely capable of driving fast transport of beads (0.53+/−0.09 instantaneous, 0.9 μm/s maximal velocity, n=54, Table 2). No motility was observed in 37 axons for other squid peptides.

### Analysis of APP-C Bead Transport

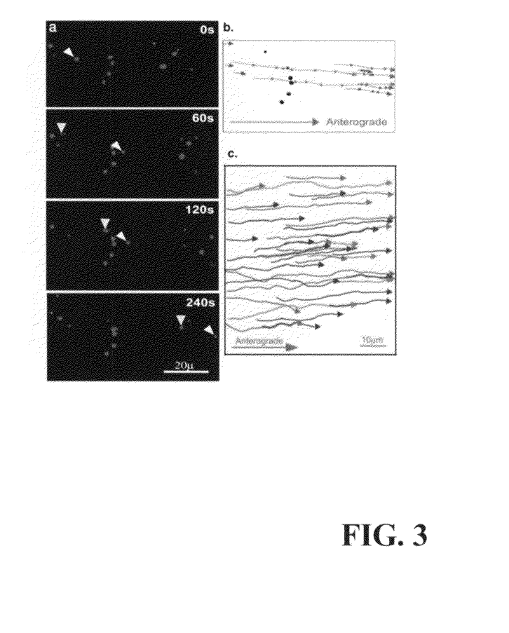

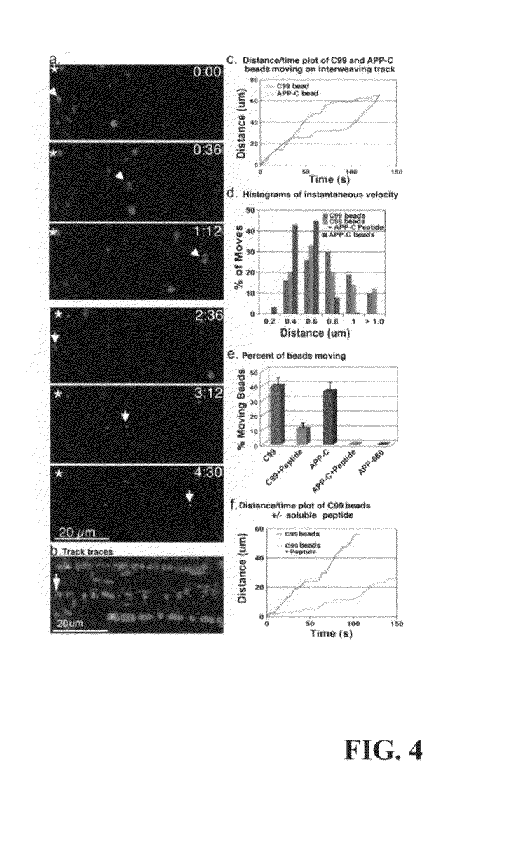

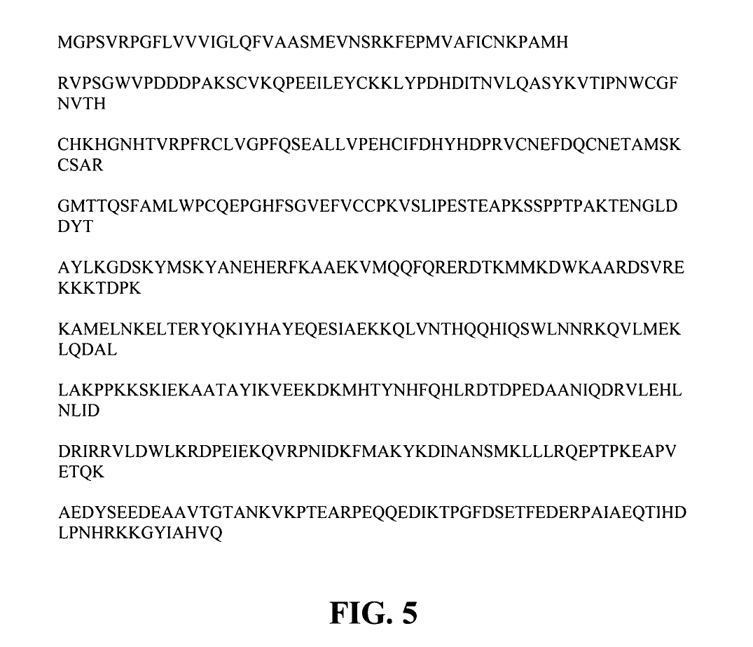

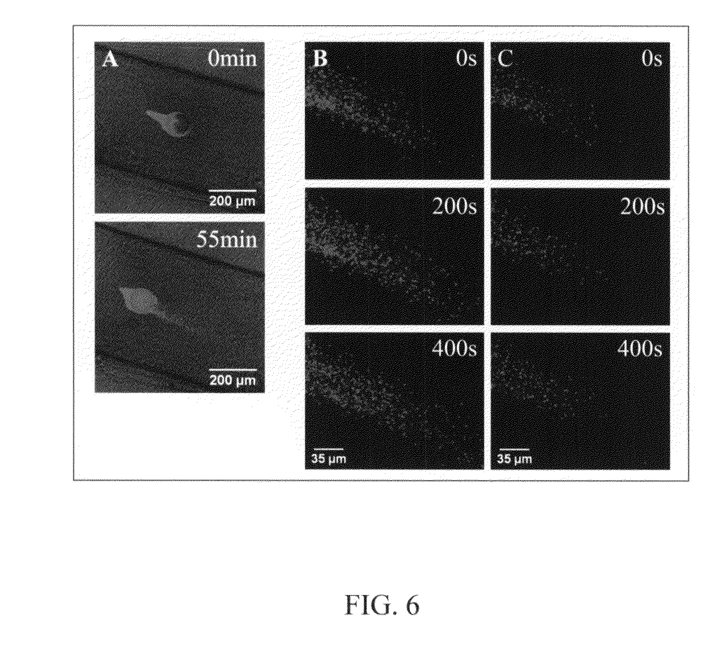

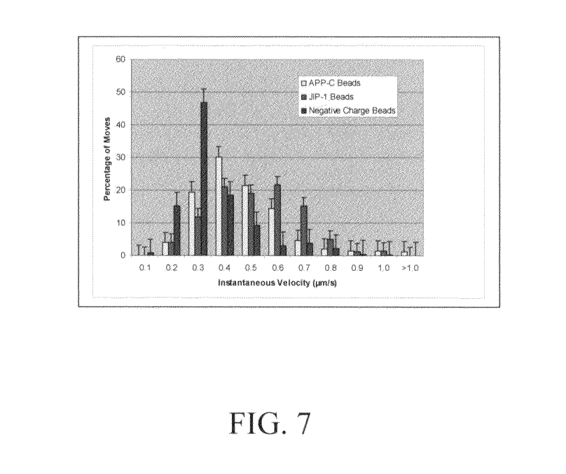

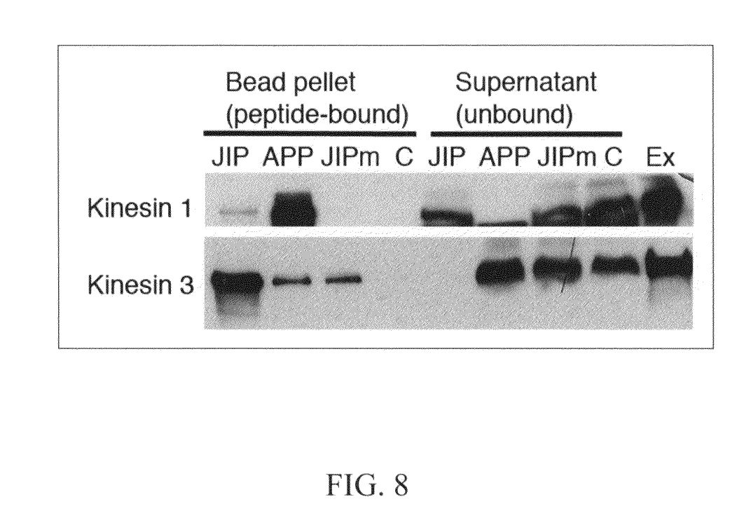

APP-C beads traveled uniquely in the anterograde direction with pauses of varying intervals but no reversals, as determined by analyzing time-lapse sequences at high magnification with intervals of 4-6 s (FIG. 3a-b). This transport was sustained for over an hour at distances >2 mm from the injection site. Tracings of multiple individual bead tracks passing through the same microscopic field over time revealed that APP-C beads traveled on interweaving parallel tracks (FIGS. 3b and c). In some cases several beads traced the same track, suggesting that beads follow each other on the same or neighboring microtubule-actin bundles (Bearer, E. L. & Reese, T. S. (1999) J Neurocytol 28, 85-98). Some beads appeared or disappeared during the sequence, as if they moved on tracks tangential to the optical section.

### C99, a Polypeptide Containing the Full-Length Cytoplasmic Domain of APP, Mediates Bead Transport

C99 contains the C-terminal 99 amino acids of APP, spanning from a short segment of the extracellular domain, the transmembrane domain and the complete cytoplasmic domain from which we derived our peptides (Table 1). Since each HSV particle with anterograde motility has on average 3,000 copies of APP (Satpute-Krishnan, P. et al., (2003) Aging Cell 2, 305-18.), we conjugated beads with equivalent amounts of C99 (3,000 copies per 100 nm bead), thereby mimicking the physiological conditions of viral transport in terms of size and APP display. In axons co-injected with C99 beads and APP-C beads, both types of beads were transported (FIG. 4a). Beads conjugated to either protein could be observed to follow each other along the same apparent track at similar velocities (FIG. 4b). Distance per time plots of two representative beads moving on super-imposable tracks shows equivalent transport rates and pauses (FIG. 4c). Calculations of instantaneous velocities of C99 and APP-C obtained from multiple bead runs further confirms similarity in movement (FIG. 4d, Table 2). Despite slightly decreased average instantaneous velocities compared to C99, APP-C beads accomplish similar distances due to longer run lengths and fewer pauses.

To test whether motility was due to an interaction of the APP-C domain with axoplasmic motors, we pre-injected axons with soluble APP-C peptide followed by co-injection of C99 and APP-C beads. Soluble peptide would be expected to compete with protein-conjugated beads for axoplasmic motors. Peptide pre-injection resulted in a 4-fold reduction of moving C99 beads and no motility of APP-C beads (FIG. 4e). Movement of a few C99 beads in peptide-injected axons confirmed that transport was still possible though diminished. When C99 beads do move in the presence of peptide, the instantaneous velocity is similar to beads without pre-injected peptide (FIG. 4d and Table 2) as would be expected if the peptide competed for the motor. In addition to reducing the proportion of beads that moved, peptide pre-injection decreased the average run length by half thus doubling the occurrence of pauses (FIG. 4f and Table 2).

Beads conjugated to a construct similar to C99 but truncated at amino acid 680 and thus lacking the APP-C domain (APP-680), were immotile in axons where other co-injected beads were transported out from the injection site.

### The APP-C Domain is Highly Conserved

The 15 aa APP-C domain is identical in APP of vertebrates (Table 3) and the GYENPTY within that sequence is identical also with invertebrates: Drosophila m., C. elegans and Loligo p. (squid). The squid sequence reported here is more similar to human than is either worm or fly, having only two differences: one amino acid substitution and one omission. The entire cytoplasmic domain of APP is almost as highly conserved as APP-C, with 100% identity among mammals and >90% between mammals and other vertebrates. These identities are higher than those for the full-length APP sequence because of the more divergent extracellular N-terminus.

### Discussion

The above results show that APP contains a 15 amino acid domain sufficient to drive the transport of beads in the giant axon of the squid. A squid APP homologue is expressed in the giant axon and encodes an identical domain equally capable of mediating transport of beads. In contrast no other peptide from the cytoplasmic domain of either human or squid APP had this activity, nor did a peptide with the same amino acids as APP-C but in jumbled order. The activity of the APPC peptide paralleled the activity of C99, a polypeptide containing the complete cytoplasmic domain. C99 also mediated transport of beads when conjugated to beads at molar amounts equivalent to that associated with motile HSV (3,000 copies of APP per particle (Satpute-Krishnan, P. et al., (2003) Aging Cell 2, 305-18)). Truncation of the terminal 15 aa removes transport capability. Pre-injection of soluble APP-C peptide abolishes APP-C bead transport and decreases the number of C99 beads moving by 75% while doubling pause length and frequency. Thus, C99 may have a higher affinity for the axoplasmic motor than APP-C. The APP-C sequence is nearly identical across species, from human to squid, worm and fly, arguing that these results uncover a highly conserved physiological interaction between APP and anterograde motor machinery. Thus, APP would be sufficient to mediate the anterograde transport of HSV during egress, and likely plays a role in endogenous membrane trafficking.

### Transport of APP-Beads Resembles Physiological Fast Axonal Transport

A test for sufficiency of any motor receptor is difficult to design inside cells, as cellular organelles contain many different proteins and may display more than one type of motor receptor. Injection of beads coated with only one type of protein into the axon allowed us to test directly whether any protein on its own were sufficient for transport independent of other organelle motor receptors. These studies do not address whether APP is necessary for cargo transport which is unlikely since there may be many ways for cargo to recruit motors, including binding to lipids (Klopfenstein, D. R., et al., (2002) Cell 109, 347-58).

The behavior of APP-C and C99 beads in axons resembled that of endogenous organelles suggesting that APP-C beads moved by normal physiologic mechanisms (Table 4). Of particular interest to us was the similarity in direction and speed of C99 beads (+0.66+/−0.08) to those of APP-associated HSV particles (+0.9+/−0.3 μm/s) in the giant axon (Satpute-Krishnan, P. et al., (2003), supra). Thus, APP is sufficient to mediate transport of 100 nm beads at concentrations similar to those found on motile virus. This supports the hypothesis that APP serves to mediate the transport of HSV during egress.

### APP Bead Transport in Context

The average and maximal velocity of moving APP-C and C99 beads is consistent with kinesin-1 mediated fast axonal transport—in vitro rates of 0.5 μm/s for soluble kinesin passively adherent to carboxylated beads (Vale, R. D., et al., (1985) Cell 40, 559-69), and 0.4-2.0 μm/s for axoplasmic organelles (Brady, S. T., et al., (1982) Science 218, 1129-31; Vale, R. D., et al., (1985) Cell 40, 449-54). Variation in transport rates of cargo carried by kinesin is attributed in part to regulation of the motor head by a cargo-binding domain in the tail (23-25) and by phosphorylations (Morfini, G., et al., (2004) Embo J 23, 2235-45; Morfini, G., et al., (2002) Embo J 21, 281-93). The GYENPTY (SEQ ID NO: 1) domain in the active peptide, APP-C, is also found in APLP1 and APLP2 (Table 3) and thus these homologues may also mediate transport. In support of this, it has been shown that a kinesin light chain, KLC1, binds APP in vitro (Kamal, A., et al., (2000) Neuron 28, 449-59) as well as APLP1 and 2 (Lazarov, O., et al., (2005) J Neurosci 25, 2386-95). These proteins may compensate for APP in knockouts where transport remains normal despite APP loss (Kamal et al., supra; Lazarov et al., supra; Zheng, H., et al., (1995) Cell 81, 525-31.). APP-kinesin interactions could occur by other mechanisms, either direct binding to heavy chains, and/or indirect binding via scaffolding proteins such as JIP1/2 that bind both kinesin and APP (Taru, H., et al., (2002) J Biol Chem 277, 20070-8; Verhey, K. J., et al., (2001) J Cell Biol 152, 959-70). In one report immuno-precipitation with anti-APP antibodies directed against the APP-C domain failed to detect an association with kinesin (Lazarov, et al., supra). Such antibodies could compete with KLC1 for binding.

No single peptide sequence, no “universal code,” common to all receptors that bind kinesin has been found. This is consistent with kinesin's ability to mediate transport of many different cargos in both the fast and slow compartments, including neurofilament (Prahlad, V., et al., (2000) J Cell Sci 113 (Pt 22), 3939-46; Terada, S. & Hirokawa, N. (2000) Curr Opin Neurobiol 10, 566-73; Shah, J. V. & Cleveland, D. W. (2002) Curr Opin Cell Biol 14, 58-62). Kinesin-1 binding partners, such as GRIP1, kinectin, JIP1/2 and JIP3 do not contain the APP-C sequence. Thus other sequences may also encode the capacity to recruit kinesin and mediate transport. Kinesin-binding domains in JIP1 (Horiuchi, D., et al., (2005) Curr Biol 15, 2137-41), and GRIP1(Setou, M., et al., (2002) Nature 417, 83-7) have been identified, and these sequences await testing in motility assays such as we report here.

The domain of APP⁶⁹⁵ that drives transport, aa 681-695, has been implicated in other cellular activities including endocytosis and transcriptional regulation (Cescato, R., et al., (2000) J Neurochem 74, 1131-9; Homayouni, R., et al., (1999) J Neurosci 19, 7507-15; Okamoto, M., et al., (2001) Neuroscience 104, 653-65; Trommsdorff, M., et al., (1998) J Biol Chem 273, 33556-60). This domain contains the NPXY motif involved in many protein-protein interactions (Homayouni, R et al., (1999), supra; Rodriguez-Boulan, E. & Musch, A. (2005) Biochim Biophys Acta 1744, 455-64). Regulation of which protein interacts with the NPXY domain of APP could occur by phosphorylations or changes in conformation. Indeed, phosphorylation of the C-terminal fragment of APP apparently affects its association with KLC1 (Inomata, H., et al., (2003) J Biol Chem 278, 22946-55) and localization to axonal processes (Muresan, Z. & Muresan, V. (2005) J Cell Biol 171, 615-25; Muresan, Z. & Muresan, V. (2005) J Neurosci 25, 3741-51). The slight differences in transport behavior of C99 compared to APP-C beads could be secondary to post-translational events on epitopes outside the APP-C domain such as phosphorylations of Thr 668 that affect localization.

The robust motility of C99 beads in the intact axon argues for a physiologic role of APP in recruitment of anterograde transport machinery inside cells. The ability of the short APP-C peptide to tag exogenous cargo for fast axonal transport has implications beyond demonstrating a role for APP in transport. This peptide sequence could be used to send exogenous molecules expressed within the neuron to the synapse. The identification of a minimal amino acid sequence that is capable of delivering exogenous cargo to the pre-synaptic terminal offers exciting possibilities, such as tagging tract tracers to delineate neuronal pathways, or tagging exogenous molecules for delivery to pre-synaptic termini for therapeutic or engineering purposes.

### EXAMPLE 2

### JIP Peptide-Coated Beads are Transported in the Axon

JIP peptide was conjugated to 100 nm fluorescent nanospheres (beads) following the protocol described in Example 1. Washed conjugated beads were injected into a freshly dissected giant squid axon and transport imaged by confocal microscopy.

A typical injection site is shown in panel (A) of FIG. 6. The red JIP beads appear at the injection site at the beginning of confocal imaging, within 3 min of injection (0 min). At approximately an hour later, a stream of beads can be seen heading towards the synapse on the right side of the injection site (lower panel, 55 min).

Higher magnification of the red JIP beads is shown in selected frames from a time-lapse series in panel (B) of FIG. 6. Glycine-quenched green beads were co-injected and although a few appear beyond the injection site, these do not change their position over the 400 sec of this video sequence.

These data demonstrate the ability of a further, JIP-based peptide to mediate anterograde transport of exogenous cargo inside the cell. JIP is thought to collaborate with APP in recruiting kinesin motors to organelle membranes. These data show that JIP can independently recruit a motor for anterograde transport.

### EXAMPLE 3

### Comparison of Velocity of JIP and APP Peptide Beads and Carboxylated Negative Charge Beads in the Giant Axon

Movements of beads conjugated to APP-C, JIP, or unconjugated (negative charge) after injection in the axon were measured using Metamorph imaging software (Molecular Devices). Average velocity of JIP beads was 20% faster than APP or negative charge. As is known, negative charge beads are transported in the axon but less frequently than peptide-conjugated beads.

The results are shown in FIG. 7, and summarized in the following table:

| | Ave velocity | Max velocity |
|---|---|---|
| Peptide | (μm/sec) | (μm/sec) |
| APP-C | 0.4 +/− 0.03 | 1.4 |
| | (n = 432) | |
| JIP-1 | 0.5 +/− 0.01 | 0.96 |
| | (n = 356) | |
| Negative charge | 0.31 +/− 0.02 | 0.94 |
| | (n = 412) | |

This data compares the instantaneous velocity of beads in the axon. The different surfaces of the beads influence the average rate of movements. In this experiment, movements between frames were measured only when beads moved into and out of the frame. This data does not take into account frequency of movement, ie pauses and run lengths. JIP moves almost twice as fast as negative charge beads, probably because it binds to a faster motor and binds more tightly.

These data show that our peptides confer selective, strong directionality and force to cargo movements.

### EXAMPLE 4

### APP-C Peptide and JIP Peptide Bind Different Motors

Rat brain extract was prepared from frozen rat brain (Pelco). Rat brain was cut from a frozen block of cortex, weighed, and solubilized in 1% Triton and buffer with sonication (3 times 1 sec bursts with a Branson sonicator set at microtip 4). After clarifying centrifugation at 15,000×g for 15 min, 10 μl of extract in pulldown buffer (Lazarov et al. 2005, supra) was layered over carboxylated beads conjugated to various peptides according to the manufacturer's instructions (Dynal).

FIG. 8 is a Western blot of peptide beads and supernatants showing that beads conjugated with APP preferentially bind to kinesin 1 heavy chain, effectively clearing it from the brain extract as demonstrated by the absence of a kinesin 1 band in the supernatant lane of the APP bead pull down (upper panel).

In contrast, JIP peptide preferentially pulls down kinesin 3 (Kif1a) from brain extract, removing all of this kinesin motor from the extract, as demonstrated by the absence of a band for kinesin 3 in the supernatant lane after JIP-bead exposure (lower panel).

In comparison APP-C and JIPm, a mutated version of JIP with a single amino acid substitution, bind only a small amount of Kinesin 3. Control beads without peptide (lanes labelled C) do not pulldown either motor.

These results show that the peptides of the present invention are selective for different anterograde motors. This makes each peptide more specific and will allow better control over intracellular targetting. For example, kinesin 1 is ubiquitously expressed and appears to carry cargo to any outbound destination. In contrast, kinesin 3 is a faster motor and selective for neurotransmitter traffick. Thus, fusions with the JIP peptide are likely to mediate faster delivery via synaptic vesicle transport system whereas APP fusions will be slower but more ubiquitously useful for sending cargo to cell surfaces.

While the present invention has been illustrated by reference to certain embodiments, it is not so limited. Indeed, various modifications of the invention in addition to those shown and described herein will become apparent to those skilled in the art from the foregoing description and fall within the scope of the appended claims.

All references cited throughout the disclosure are hereby expressly incorporated by reference.

TABLE 1
Diagram of APP showing location of peptide sequences and
C99 construct used in this study

| Peptide | Position | | SEQ ID NO |
|---|---|---|---|
| APP-N | 44-63 | HMNCQNGKWDSDPSGTKTCI | 2 |
| hJMD | 647-665 | KKKQYTSIHHGVVEVDA | 3 |
| sJMD | 565-582 | KRRTQRQRVTHGFVEVDP | 4 |
| hC^{mid} | 666-681 | AVTPEERHLSKMQQN | 5 |
| sC^{mid} | 583-597 | AASPEERHVANMQMS | 6 |
| hAPP-C | 681-695 | GYENPTYKFFEQMQN | 7 |
| sAPP-C | 598-612 | GYENPTYKYFE-MQN | 8 |
| hAPP-C^{j} | 681-695 | GEPYFEMNYNTKFQQ | 9 |

h = human;
s = squid sequences.
Numbers are for human neuronal isoformAPP⁶⁹⁵ (NCBI UniGene Hs.434980) or squid APP (Acc. No. DQ913735).

TABLE 2
Comparisons of velocities

| | | Max. |
|---|---|---|
| Cargo type | Ave. Velocity (μm/s) | Velocity (μm/s) |
| Beads conjugated to: | | |
| APP-C (human/rat) | 0.41 +/− 0.07 (227) | 0.9 |
| APP-C (squid) | 0.53 +/− 0.09 (54) | 0.9 |
| C99 | 0.66 +/− 0.08 (194) | 1.46 |
| Beads, negatively charged | | |
| in squid axons¹ | 0.078 (70) | 0.4 |
| in crab axons² | 0.3 (22) | 0.6 |
| Mitochondria in squid axons⁵ | 0.2 +/− 0.08 (45) | 0.36 |
| Endogenous organelles | 0.4-2.0 | 5.0 |
| in squid axoplasm^{3,4} | | |
| HSV in squid axons | | |
| Anterograde⁵ | 0.9 +/− 0.3 (73) | 1.23 |
| Retrograde⁶ | 2.2 +/− 0.26 (76) | 2.7 |

¹Terasaki et al., (1995) Proc Natl Acad Sci USA 92, 11500-3
²Calculated from data presented in Adams and Bray, 1983.
³Vale et al., (1985) Cell 40, 559-69.
⁴Allen et al., 1982.
⁵Satpute-Krishnan, DeGiorgis and Bearer, (2003) Aging Cell 2, 305-18.
⁶Bearer et al., 2000

TABLE 3
Conservation of APP-C and APP

| | | % identity |
|---|---|---|
| | APP-C aa sequence | C-terminus/full |
| Species (Acc. number) | line-up | length |
| Homo sapiens (P05067) | GYENPTYKFFEQMQN | 100/100 |
| Macaca f. (A49795 | GYENPTYKFFEQMQN | 100/99 |
| Rattus r. (S00550) | GYENPTYKFFEQMQN | 100/97 |
| Mus m. (A27485) | GYENPTYKFFEQMQN | 100/97 |
| Xenopus l. (JH0773) | GYENPTYKFFEQMQN | 96/87 |
| Danio r. (Q90W28) | GYENPTYKFFEQMQN | 92/74 |
| Takifugu r. (O93279) | GYENPTYKFFEQMQN | 92/71 |
| Loligo p. (squid) (TBA) | GYENPTYKYFE-MQN | 64/38 |
| Drosophila m. (A32758) | GYENPTYKYFE-VKE | 53/30 |
| C. elegans (T15795) | GYENPTYSFFD-SKA | 48/36 |
| Homo sapiens | | |
| APLP1(AAH12889) | GYENPTYRFLEERP | 53/41 |
| APLP2 (AAH12889) | GYENPTYKYLEQMQI | 68/52 |

What is claimed is:

1. A peptide of the formula GYENPTYX1X2X3X4X5X6X7X8 (SEQ ID NO: 13), wherein
X1 is K, S, or R;
X2 is F or Y;
X3 is F or L;
X4 is E or D;
X5 is missing or is Q or E;
X6 is M, V, S, or R;
X7 is Q, K or P;
X8 is missing or is N, E, A or I.

2. The peptide of claim 1 wherein X1 is K, X2 is F, X3 is F, X4 is E, X5 is Q, X6 is M, X7 is Q and X8 is N.

3. The peptide of claim 1 wherein X1 is K, X2 is Y, X3 is F, X4 is E, X5 is missing, X6 is M, X7 is Q and X8 is N.

4. The peptide of claim 1, claim 2, or claim 3 conjugated to an exogeneous or endogeneous cargo.

5. The peptide of claim 4 wherein said exogenous cargo is selected from the group consisting of a cell, a peptide, a polypeptide, and a non-peptide small molecule.

6. The peptide of claim 5 wherein said cell is a non-brain cell.

7. The peptide of claim 4 wherein said exogenous cargo is a brain permeable molecule or an adhesion molecule.

8. A method for facilitating anterograde neuronal transport of a cargo comprising conjugating said cargo to a peptide of about 12 to about 17 amino acids, comprising the amino acid sequence of GYENPTY (SEQ ID NO: 1), and delivering the conjugate obtained along a neuronal pathway to a synapse or the surface of a cell.

9. The method of claim 8 wherein said cargo is an endogenous cargo or exogeneous cargo.

10. The method of claim 9 wherein said exogenous cargo is selected from the group consisting of a cell, a peptide, a polypeptide, and a non-peptide small molecule.

11. The method of claim 10 wherein said cell is a non-brain cell.

12. The method of claim 9 wherein said exogenous cargo is a brain permeable molecule or an adhesion molecule.

13. The method of claim 8 wherein said neuronal transport is axonal transport.

14. The method of claim 8 wherein said peptide has the formula

| | |
|---|---|
| GYENPTYX1X2X3X4X5X6X7X8, | (SEQ ID NO: 13) |

wherein
X1 is K, S, or R;
X2 is F or Y;
X3 is F or L;
X4 is E or D;
X5 is missing or is Q or E;
X6 is M, V, S, or R;
X7 is Q, K or P;
X8 is missing or is N, E, A or I.

15. The method of claim 14 wherein in SEQ ID NO: 13 X1 is K, X2 is F, X3 is F, X4 is E, X5 is Q, X6 is M, X7 is Q and X8 is N.

16. The method of claim 14 wherein in SEQ ID NO: 13 X1 is K, X2 is Y, X3 is F, X4 is E, X5 is missing, X6 is M, X7 is Q and X8 is N.

17. A method for the treatment of a disease or condition benefiting from the facilitation of neuronal transport in a mammalian subject, comprising administering to said mammalian subject an effective amount of a conjugate of a cargo and a peptide of about 12 to about 17 amino acids, comprising the amino acid sequence of GYENPTY (SEQ ID NO: 1).

18. The method of claim 17 wherein said peptide has the formula

| | |
|---|---|
| GYENPTYX1X2X3X4X5X6X7X8, | (SEQ ID NO: 13) |

wherein
X1 is K, S, or R;
X2 is F or Y;
X3 is F or L;
X4 is E or D;
X5 is missing or is Q or E;
X6 is M, V, S, or R;
X7 is Q, K or P;
X8 is missing or is N, E, A or I.

19. The method of claim 18 wherein in SEQ ID NO: 13 X1 is K, X2 is F, X3 is F, X4 is E, X5 is Q, X6 is M, X7 is Q and X8 is N.

20. The method of claim 18 wherein in SEQ ID NO: 13 X1 is K, X2 is Y, X3 is F, X4 is E, X5 is missing, X6 is M, X7 is Q and X8 is N.

21. The method of claim 17 wherein said disease or condition is a neurodegenerative disease.

22. The method of claim 17 wherein said disease or condition is selected from the group consisting of Alzheimer's disease, Huntington's disease, amyotrophic lateral sclerosis (ALS), siliary dyskinesias, retinitis pigmentosa, and Creutzfeldt-Jacob disease.

23. A method for the treatment of a disease or condition benefiting from the inhibition of neuronal transport in a mammalian subject, comprising administering to said mammalian subject an effective amount of an inhibitor of the cytoplasmic C-terminus of the amyloid precursor protein (APP-C) of said mammalian subject.

24. The method of claim 33 wherein said inhibitor comprises the amino acid sequence of GYENPTY (SEQ ID NO: 1).

25. The method of claim 23 wherein said inhibitor comprises a peptide of the formula

| | |
|---|---|
| GYENPTYX1X2X3X4X5X6X7X8, | (SEQ ID NO: 13) |

wherein
X1 is K, S, or R;
X2 is F or Y;
X3 is F or L;
X4 is E or D;
X5 is missing or is Q or E;
X6 is M, V, S, or R;
X7 is Q, K or P;
X8 is missing or is N, E, A or I.

26. The method of claim 25 wherein in SEQ ID NO: 13 X1 is K, X2 is F, X3 is F, X4 is E, X5 is Q, X6 is M, X7 is Q and X8 is N.

27. The method of claim 25 wherein in SEQ ID NO: 13 X1 is K, X2 is Y, X3 is F, X4 is E, X5 is missing, X6 is M, X7 is Q and X8 is N.

28. The method of claim 23 wherein said inhibitor is a small molecule binding to said APP-C.

29. The method of claim 23 wherein said inhibitor is a non-peptide small molecule binding to said APP-C.

30. The method of claim 23 wherein said disease or condition is Down's syndrome.

31. A pharmaceutical composition comprising a peptide of the formula

| | |
|---|---|
| GYENPTYX1X2X3X4X5X6X7X8, | (SEQ ID NO: 13) |

wherein
X1 is K, S, or R;
X2 is F or Y;
X3 is F or L;
X4 is E or D;
X5 is missing or is Q or E;
X6 is M, V, S, or R;
X7 is Q, K or P;
X8 is missing or is N, E, A or I,
in admixture with a pharmaceutically acceptable ingredient.

32. A pharmaceutical composition comprising a conjugate of a peptide of the formula

| | |
|---|---|
| GYENPTYX1X2X3X4X5X6X7X8, | (SEQ ID NO: 13) |

wherein
X1 is K, S, or R;
X2 is F or Y;
X3 is F or L;
X4 is E or D;
X5 is missing or is Q or E;
X6 is M, V, S, or R;
X7 is Q, K or P;
X8 is missing or is N, E, A or I,
and a cargo, in a mixture with a pharmaceutically acceptable ingredient.

34. A peptide of 12 to 17 amino acids comprising the peptide of FVE YTC PTE DIY LE (SEQ ID NO: 14).

35. The peptide of FVE YTC PTE DIY LE (SEQ ID NO: 14).

36. The peptide of claim 34 or claim 35 conjugated to an exogeneous or endogeneous cargo.

37. The peptide of claim 36 wherein said exogenous cargo is selected from the group consisting of a cell, a peptide, a polypeptide, and a non-peptide small molecule.

38. The peptide of claim 37 wherein said cell is a non-brain cell.

39. The peptide of claim 36 wherein said exogenous cargo is a brain permeable molecule or an adhesion molecule.

40. A method for facilitating anterograde neuronal transport of a cargo comprising conjugating said cargo to a peptide of claim 34 or claim 35, and delivering the conjugate obtained along a neuronal pathway to a synapse or the surface of a cell.

41. A method for the treatment of a disease or condition benefiting from the facilitation of neuronal transport in a mammalian subject, comprising administering to said mammalian subject an effective amount of a conjugate of a cargo and a peptide of claim 34 or 35.

42. The method of claim 41 wherein said disease or condition is selected from the group consisting of Alzheimer's disease, Huntington's disease, amyotrophic lateral sclerosis (ALS), siliary dyskinesias, retinitis pigmentosa, and Creutzfeldt-Jacob disease.

43. A pharmaceutical composition comprising the peptide of claim 34 or claim 35, in admixture with a pharmaceutically acceptable ingredient.

44. A method of delivery of an exogeneous epitope to the surface of a target cell comprising conjugating said epitope to a peptide of claim 1, 34 or 35, and delivering the conjugate formed to the cell surface by anterograde transport.

45. The method of claim 44 wherein said target cell is a cancer cell.

46. The method of claim 46 wherein said exogenous epitope makes the cancer cell susceptible to an anti-cancer agent.

47. The method of claim 46 further comprising the step of exposing said cell to said anti-cancer agent.


SEQUENCE LISTING (25 sequences)

SEQ ID NO: 1 - protein, 7 aa, Artificial Sequence
  Description of Artificial Sequence Synthetic peptide
GYENPTY

SEQ ID NO: 2 - protein, 20 aa, Homo sapiens
HMNCQNGKWDSDPSGTKTCI

SEQ ID NO: 3 - protein, 17 aa, Homo sapiens
KKKQYTSIHHGVVEVDA

SEQ ID NO: 4 - protein, 18 aa, Loligo pealei
KRRTQRQRVTHGFVEVDP

SEQ ID NO: 5 - protein, 15 aa, Homo sapiens
AVTPEERHLSKMQQN

SEQ ID NO: 6 - protein, 15 aa, Loligo pealei
AASPEERHVANMQMS

SEQ ID NO: 7 - protein, 15 aa, Homo sapiens
GYENPTYKFFEQMQN

SEQ ID NO: 8 - protein, 14 aa, Loligo pealei
GYENPTYKYFEMQN

SEQ ID NO: 9 - protein, 15 aa, Homo sapiens
GEPYFEMNYNTKFQQ

SEQ ID NO: 10 - protein, 520 aa, Loligo pealei
MGPSVRPGFLVVVIGLQFVAASMEVNSRKFEPMVAFICNKPAMHRVPSGWVPDDDPAKSCVKQPEEILEYCKKLYPDHDITNVLQASYKVTIPNWCGFNVTHCHKHGNHTVRPFRCLVGPFQSEALLVPEHCIFDHYHDPRVCNEFDQCNETAMSKCSARGMTTQSFAMLWPCQEPGHFSGVEFVCCPKVSLIPESTEAPKSSPPTPAKTENGLDDYTAYLKGDSKYMSKYANEHERFKAAEKVMQQFQRERDTKMMKDWKAARDSVREKKKTDPKKAMELNKELTERYQKIYHAYEQESIAEKKQLVNTHQQHIQSWLNNRKQVLMEKLQDALLAKPPKKSKIEKAATAYIKVEEKDKMHTYNHFQHLRDTDPEDAANIQDRVLEHLNLIDDRIRRVLDWLKRDPEIEKQVRPNIDKFMAKYKDINANSMKLLLRQEPTPKEAPVETQKAEDYSEEDEAAVTGTANKVKPTEARPEQQEDIKTPGFDSETFEDERPAIAEQTIHDLPNHRKKGYIAHVQ

SEQ ID NO: 11 - protein, 47 aa, Loligo pealei
LKRRTQRQRVTHGFVEVDPAASPEERHVANMQMGYENPTYKYFEMQN

SEQ ID NO: 12 - protein, 47 aa, Homo sapiens
LKKKQYTSIHHGVVEVDAAVTPEERHLSKMQQGYENPTYKFFEQMQN

SEQ ID NO: 13 - protein, 15 aa, Artificial Sequence
  Description of Artificial Sequence Synthetic peptide
GYENPTYXXXXXXXX

SEQ ID NO: 14 - protein, 14 aa, Artificial Sequence
  Description of Artificial Sequence Synthetic peptide
FVEYTCPTEDIYLE

SEQ ID NO: 15 - protein, 47 aa, Rattus sp.; identical to SEQ ID NO: 12

SEQ ID NO: 16 - protein, 15 aa, Macaca sp.
GYENPTYKFFEQMQN

SEQ ID NO: 17 - protein, 15 aa, Rattus rattus
GYENPTYKFFEQMQN

SEQ ID NO: 18 - protein, 15 aa, Mus musculus
GYENPTYKFFEQMQN

SEQ ID NO: 19 - protein, 15 aa, Xenopus laevis
GYENPTYKFFEQMQN

SEQ ID NO: 20 - protein, 15 aa, Danio rerio
GYENPTYKFFEQMQN

SEQ ID NO: 21 - protein, 15 aa, Takifugu rubripes
GYENPTYKFFEQMQN

SEQ ID NO: 22 - protein, 14 aa, Drosophila melanogaster
GYENPTYKYFEVKE

SEQ ID NO: 23 - protein, 14 aa, Caenorhabditis elegans
GYENPTYSFFDSKA

SEQ ID NO: 24 - protein, 14 aa, Homo sapiens
GYENPTYRFLEERP

SEQ ID NO: 25 - protein, 15 aa, Homo sapiens
GYENPTYKYLEQMQI